(19) United States
(12) Patent Application Publication
(10) Pub. No.: US 2022/0370507 A1
(43) Pub. Date: Nov. 24, 2022

(54) ENDOTHELIAL AND SMOOTH MUSCLE LIKE TISSUE PRODUCED FROM URINE CELLS AND USES RELATED THERETO

(71) Applicant: EMORY UNIVERSITY, Atlanta, GA (US)

(72) Inventors: Young-Sup YOON, Atlanta, GA (US); Young-Doug SOHN, Atlanta, GA (US); Sang-Ho LEE, Atlanta, GA (US)

(21) Appl. No.: 17/761,876

(22) PCT Filed: Jul. 10, 2020
(86) PCT No.: PCT/US2020/041537

§ 371 Date: Mar. 18, 2022

Related U.S. Application Data
(60) Provisional application No. 62/903,154, filed on Sep. 20, 2019.

(51) Int. Cl. A61K 35/44; A61L 27/36; C12N 5/071

(52) U.S. Cl. CPC A61K 35/44; A61L 27/3625; C12N 5/0691; C12N 2500/38; C12N 2501/11; C12N 2501/165; C12N 2501/91; C12N 2506/25; C12N 2513/00; C12N 2710/10343; C12N 2501/39; C12N 2500/32; C12N 2501/115; C12N 2500/34; C12N 2500/46

(57) ABSTRACT

This disclosure relates to endothelial and smooth muscle like vascular tissue produced from urine cells. In certain embodiments, the disclosure relates to methods of producing endothelial and smooth muscle like vascular tissue by exposing urine derived cells with ETV2 in a first growth media under conditions such that the cells are modified to form a pool of cells expressing increased levels of endothelium surface markers and thereafter exposing the pool of cells to a second growth media under conditions such that the cells are modified to form tissue containing cells expressing increased levels of smooth muscle surface markers in addition to the endothelium surface markers. In certain embodiments, the disclosure relates to using cells and tissues reported herein for the treatment of vascular, cardiac, and wound healing indications.

### CROSS-REFERENCE TO RELATED APPLICATIONS

This application claims the benefit of U.S. Provisional Application No. 62/903,154 filed Sep. 20, 2019. The entirety of this application is hereby incorporated by reference for all purposes.

### STATEMENT REGARDING FEDERALLY FUNDED RESEARCH

This invention was made with government support under DK108245, HL127759, and HL129511 awarded by the National Institutes of Health. The government has certain rights in the invention.

### INCORPORATION-BY-REFERENCE OF MATERIAL SUBMITTED AS A TEXT FILE VIA THE OFFICE ELECTRONIC FILING SYSTEM (EFS-WEB)

The Sequence Listing associated with this application is provided in text format in lieu of a paper copy and is hereby incorporated by reference into the specification. The name of the text file containing the Sequence Listing is 19161PCT_ST25.txt. The text file is 11 KB, was created on Jul. 9, 2020, and is being submitted electronically via EFS-Web.

### BACKGROUND

Ischemic cardiovascular diseases are major causes of morbidity and mortality in industrialized country. Risk factor management, pharmacological treatment, and surgical revascularization are current therapeutic options, but are not always effective when permanent loss of vessels occurs. Despite significant efforts made over the last several decades, treating patients with ischemic cardiac and vascular disease remains a challenge. Thus, there is a significant need to develop therapies that restore blood supply in the host organs through neovascularization.

Endothelial cells (ECs) are a key element of vasculature and are indispensable for repairing injured or ischemic tissues. Over the years, there have been may attempts to generate ECs for use in cell therapy. Despite early enthusiasm, adult stem or progenitor cells were found to have minimal endothelial transdifferentiation potential. Embryonic stem cells (ESCs) and induced pluripotent stem cells (iPSCs) emerged as promising alternatives; however, problems such as tumorigenic potential or inefficient cell production have limited their clinical application. Thus, there is a need to identify improvements.

Lee et al. report reprogramming of human dermal fibroblasts into endothelial cells using ER71/ETV2.

Veldman et al. report transdifferentiation of fast skeletal muscle into functional endothelium in vivo by transcription factor Etv2. PLoS Biol, 2013, 11(6): e1001590.

Bharadwaj et al. report multipotential differentiation of human urine-derived stem cells. Stem Cells 2013, 31:1840-1856.

References cited here are not an admission of prior art.

### SUMMARY

This disclosure relates to endothelial and smooth muscle like vascular tissue produced from urine cells. In certain embodiments, the disclosure relates to methods of producing endothelial and smooth muscle like vascular tissue by exposing urine derived cells with ETV2 in a first growth media under conditions such that the cells are modified to form a pool of cells expressing increased levels of endothelium surface markers and thereafter exposing the pool of cells to a second growth media under conditions such that the cells are modified to form tissue containing cells expressing increased levels of smooth muscle surface markers in addition to the endothelium surface markers. In certain embodiments, the disclosure relates to using cells and tissues reported herein for the treatment of vascular, cardiac, and wound healing indications.

In certain embodiments, this disclosure relates to methods of producing endothelial and smooth muscle like vascular tissue comprising: i) concentrating urine cells from a subject; ii) replicating the concentrated urine cells in a first growth media comprising, a) EGF, b) hydrocortisone, c) epinephrine and d) human serum or animal serum; providing purified concentrated urine derive cells; iii) exposing the purified concentrated urine derive cells to ETV2; iv) culturing the purified concentrated urine derive cells in the first growth media providing endothelial like urine derived cells; v) culturing the endothelial like urine derive cells in a second growth media comprising: a) EGF, b) VEGFA, c) bFGF, d) heparin, e) L-ascorbic acid, and d) human serum or animal serum; providing endothelial and smooth muscle like vascular tissue. In certain embodiments, the human serum is from a subject to be treated with or implanted with the vascular tissue.

In certain embodiments, exposing the purified concentrated urine derive cells to ETV2 is by mixing the purified concentrated urine derived cells with a recombinant virus that infects the purified concentrated urine derive cells, comprises a gene encoding ETV2, and expresses ETV2 after infection. In certain embodiments, the recombinant virus is an adenovirus or lentivirus.

In certain embodiments, the first or second growth media comprises glucose, amino acids, and vitamins, glutamine, and sodium pyruvate.

In certain embodiments, methods disclosed herein further comprise the step of folding the endothelial and smooth muscle like vascular tissue into a three-dimensional structure. In certain embodiments, methods disclosed herein further comprise implanting the endothelial and smooth muscle like vascular tissue into the subject.

In certain embodiments, implanting the endothelial like and smooth muscle like vascular tissue is by contacting the endothelial and smooth muscle like vascular tissue with a vein, artery, capillary, or heart muscle.

In certain embodiments, the disclosure relates to methods of producing endothelial or endothelial like cells comprising exposing expanded urine cells comprising a recombinant vector encoding ETV2 in operable combination with a promotor to a stimulus of the promotor under conditions such that ETV2 is formed in the cells and the expanded urine cells are modified to form a pool of cells expressing increased levels of endothelium surface markers, wherein the surface markers are KDR and CDH5, thereby providing endothelial like cells.

In certain embodiments, the pool of cells expresses increased levels of the surface markers KDR and CDH5. In certain embodiments, the pool of cells expresses increased levels of the surface markers PECAM1 and TEK.

In certain embodiments, urine cells or expanded cells do or do not comprise a recombinant vector that encodes ERG or FLI1 or do or do not comprise a recombinant vector that encodes FOXC2, MEF2C, SOX17, NANOG, or HEY1.

In certain embodiments, urine cells or expanded urine derived cells are or are not in contact with a medium comprising a TGFβ inhibitor.

In certain embodiments, the methods disclosed herein further comprising the step of purifying the pool of purified urine derived cells by selecting cells that express KDR providing purified pool of KDR urine derived cells. In certain embodiments, the methods disclosed herein further comprising the step of purifying the pool of purified urine derived cells by selecting cells that do not express KDR providing purified pool of KDR negative urine derived cells.

In certain embodiments, the disclosure relates to compositions comprising cells made by the processes disclosed herein.

In certain embodiments, the methods disclosed herein further comprise the step of generating endothelial like cells comprising contacting the cells produced herein with valproic acid.

In certain embodiments, the methods disclosed herein further comprises the step of generating a modified pool of cells comprising contacting the cells produced herein with the promotor stimulus.

In certain embodiments, the methods disclosed herein further comprise the step of generating a modified pool of cells comprising contacting the cells of produced herein with collagen.

In certain embodiments, the disclosure relates to methods of treating or preventing a skin condition, disease, an injury, contusion, open-wound, laceration, vascular condition, disease, heart condition, disease, atherosclerosis, coronary artery disease, or ischemia comprising administering an effective amount of cells or tissues produced herein to a subject in need thereof.

### BRIEF DESCRIPTION OF THE DRAWINGS

FIG. 1 illustrates a method for generating vascular mimetic tissue. Human urine cells are collected by centrifugation of urine. Cell pellets are suspended in growth media (urine cell growth media: 10% fetal bovine serum containing Dulbecco's modified Eagle's medium (DMEM), DMEM/nutrient mixture F-12 (DMEM/F-12 contains no proteins, lipids, or growth factors). Growth factor EGF, hydrocortisone, and epinephrine are added. The cell suspension is incubated at 37° C., CO₂ (5%) incubator for 14 days. Select cells replicate to form colonies. The reprogramming of replicated urine cells is initiated by infection with an adenovirus that encodes ETV2. The urine derived cells grow for 24 hours. Thereafter, the media is changed to include VEGFA, EGF, bFGF, heparin, and vitamin C.

FIG. 2 shows data indicating endothelial genes are significantly induced in ETV2 treated replicated urine cells.

FIG. 3 shows the tubular network structure on vascular mimetic tissue.

FIG. 4 shows data indicating the non-EC population (KDR negative) is significantly enriched with smooth muscle cells specific genes.

FIG. 5 illustrates the fabrication of vascular mimetic tissue.

FIG. 6 illustrates a sequence comparison of ETV2, isoform 1, for Human (H. sapiens, Query, NCBI Accession Number NP 055024.2) and Mouse (M. musculus, Subject, NCBI Accession Number NP 031985.2). 232/344 (67% identities), 250/344 (73% positive), and 11/344 (3% gaps).

### DETAILED DISCUSSION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of medicine, organic chemistry, biochemistry, molecular biology, pharmacology, and the like, which are within the skill of the art. Such techniques are explained fully in the literature.

It must be noted that, as used in the specification and the appended claims, the singular forms “a,” “an,” and “the” include plural referents unless the context clearly dictates otherwise. Thus, for example, reference to “a support” includes a plurality of supports. In this specification and in the claims that follow, reference will be made to a number of terms that shall be defined to have the following meanings unless a contrary intention is apparent.

As used in this disclosure and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) have the meaning ascribed to them in U.S. patent law in that they are inclusive or open-ended and do not exclude additional, unrecited elements or method steps. For example, the term “comprising” in reference to an oligonucleotide having a nucleic acid sequence refers to an oligonucleotide that may contain additional 5′ (5′ terminal end) or 3′ (3′ terminal end) nucleotides, i.e., the term is intended to include the oligonucleotide sequence within a larger nucleic acid. “Consisting essentially of” or “consists of” or the like, when applied to methods and compositions encompassed by the present disclosure refers to compositions like those disclosed herein that exclude certain prior art elements to provide an inventive feature of a claim, but which may contain additional composition components or method steps, etc., that do not materially affect the basic and novel characteristic(s) of the compositions or methods, compared to those of the corresponding compositions or methods disclosed herein.

The term, “serum” refers to the blood product obtained when blood of an animal is allowed to clot, and the clot is separated from the blood. Fetal bovine serum is derived from of blood from a bovine fetus after the fetus is removed from a slaughtered cow.

As used herein, a “growth medium” or “media” refers to a composition that contains components that facilitate cell maintenance and growth through protein biosynthesis, such as vitamins, amino acids, inorganic salts, a buffer, and a fuel, e.g., acetate, succinate, a saccharide and/or optionally nucleotides. Additionally, a growth media may contain phenol red as a pH indication. Components in the growth medium may be derived from blood serum or the growth medium may be serum-free. The growth medium may optionally be supplemented with albumin, lipids, insulin and/or zinc, transferrin or iron, selenium, ascorbic acid, and an antioxidant such as glutathione, 2-mercaptoethanol or 1-thioglycerol. Other contemplated components contemplated in a growth medium include ammonium metavanadate, cupric sulfate, manganous chloride, ethanolamine, and sodium pyruvate. Minimal Essential Medium (MEM) is a term of art referring to a growth medium that contains calcium chloride, potassium chloride, magnesium sulfate, sodium chloride, sodium phosphate and sodium bicarbonate), essential amino acids, and vitamins: thiamine (vitamin B1), riboflavin (vitamin B2), nicotinamide (vitamin B3), pantothenic acid (vitamin B5), pyridoxine (vitamin B6), folic acid (vitamin B9), choline, and myo-inositol (originally known as vitamin B8). Various growth mediums are known in the art. Dulbecco's modified Eagle's medium (DMEM) is a growth medium which contains additional components such as glycine, serine and ferric nitrate with increased amounts of vitamins, amino acids, and glucose as indicated in Table 1 below.

TABLE 1
Composition of Dulbecco's modified Eagle's medium

| Components | Concentration (mg/L) |
|---|---|
| Amino Acids | |
| Glycine | 30.0 |
| L-Arginine hydrochloride | 84.0 |
| L-Cystine 2HCl | 63.0 |
| L-Glutamine | 584.0 |
| L-Histidine hydrochloride-H2O | 42.0 |
| L-Isoleucine | 105.0 |
| L-Leucine | 105.0 |
| L-Lysine hydrochloride | 146.0 |
| L-Methionine | 30.0 |
| L-Phenylalanine | 66.0 |
| L-Serine | 42.0 |
| L-Threonine | 95.0 |
| L-Tryptophan | 16.0 |
| L-Tyrosine disodium salt dihydrate | 104.0 |
| L-Valine | 94.0 |
| Vitamins | |
| Choline chloride | 4.0 |
| D-Calcium pantothenate | 4.0 |
| Folic Acid | 4.0 |
| Niacinamide | 4.0 |
| Pyridoxine hydrochloride | 4.0 |
| Riboflavin | 0.4 |
| Thiamine hydrochloride | 4.0 |
| i-Inositol | 7.2 |
| Inorganic Salts | |
| Calcium Chloride (CaCl2) (anhyd.) | 200.0 |
| Ferric Nitrate (Fe(NO3)3″9H2O) | 0.1 |
| Magnesium Sulfate (MgSO4) (anhyd.) | 97.67 |
| Potassium Chloride (KCl) | 400.0 |
| Sodium Bicarbonate (NaHCO3) | 3700.0 |
| Sodium Chloride (NaCl) | 6400.0 |
| Sodium Phosphate monobasic (NaH2PO4—H2O) | 125.0 |
| Other Components | |
| Phenol Red | 15.0 |

Ham's F-12 medium has high levels of amino acids, vitamins, and other trace elements. Putrescine and linoleic acid are included in the formulation. See Table 2 below.

TABLE 2
Composition of the Ham's F-12 medium

| | Concentration |
|---|---|
| Substance | (mg/L) |
| NaCI | 7599 |
| KCI | 223.6 |
| Na2HPO4 | 142 |
| CaCI2•2H2O | 44 |
| MgCl2 | 122 |
| FeSO4•7H2O | 0.834 |
| CuSO4•5H2O | 0.00249 |
| ZnSO4•7H2O | 0.863 |
| D-glucose | 1802 |
| Na-pyruvate | 110 |
| Phenol red | 1.2 |
| NaHCO3 | 1176 |
| L-alanine | 9 |
| L-arginine•HCl | 211 |
| L-asparagine 13.2 | 13.2 |
| L-aspartic acid | 13.3 |
| L-cysteine•HCI | 31.5 |
| L-glutamine | 146 |
| L-glutamic acid | 14.7 |
| Glycine | 7.5 |
| L-histidine•HCI•H2O | 21 |
| L-isoleucine | 4 |
| L-leucine | 13 |
| L-lysine•HCI | 36.5 |
| L-methionine | 4.47 |
| L-phenylalanine | 5 |
| L-proline | 34.5 |
| L-serine | 10.5 |
| L-threonine | 12 |
| L-tryptophan | 2 |
| L-tyrosine | 5.4 |
| L-valine | 11.7 |
| Biotin | 0.0073 |
| D-Ca-pantothenate | 0.48 |
| Choline chloride | 14 |
| Folic acid | 1.3 |
| Myo-inositol | 18 |
| Nicotinic acid amid | 0.037 |
| Pyridoxin•HCI | 0.062 |
| Riboflavin | 0.038 |
| Thiamine•HCI | 0.34 |
| Vitamin B12 | 1.36 |
| Hypoxanthine | L 4.1 |
| Thymidine | 0.73 |
| Lipoic acid | 0.21 |
| Linoleic acid | 0.084 |
| Putrescine•2HCI | 0.161 |

In certain embodiments, the disclosure contemplates a growth media disclosed herein using a mixture of DMEM and F-12 medium which is a 1:1 mixture of DMEM and Ham's F-12. The optimal carbon dioxide required for DME is 10% and for F-12 is 5%. Since this medium is a mixture, the optimal carbon dioxide concentration is typically 5% to 8%.

As used herein, “heparin” refers to an anticoagulant polymer with variably sulfated repeating disaccharide units. Common disaccharide units are composed of a 2-O-sulfo-α-L-iduronic acid and 2-deoxy-2-sulfamido-α-D-glucopyranosyl-6-O-sulfate.

The terms, “ETS translocation variant 2” and “ETV2” refer to a transcription factor involve in hematopoietic and vascular development. ETV2 deficiency in mice leads to a complete block in hematopoietic and vascular formation and embryonic lethality. Human recombinant ETV2 is a commercially available protein having the NCBI Reference Sequence: NP 055024.2 (SEQ ID NO: 1). Adenovirus encoding ETV2 are also commercially available for expressing ETV2 mRNA, see NCBI Reference Sequence: NM 014209.4.

In certain embodiments, the disclosure contemplates exposing urine derived cells with ETV2 under conditions such that ETV2 is produced optionally as a C-terminal or N-terminal fusion with a cell-penetrating peptide (CPP), e.g., poly-arginine, i.e., and contacting the ETV2 fusion with urine derived cells. Warren et al. report reprogramming to pluripotency and directed differentiation of human cells with synthetic modified mRNA. Cell stem cell, 2010, 7:618-630. In certain embodiments, the disclosure contemplates exposing urine derived cells with mRNA of ETV2, e.g., delivery of mRNA into the cells by using electroporation or by complexing the RNA with a cationic vehicle to facilitate uptake by endocytosis.

The terms, “Epidermal growth factor” and “EGF” refer to a protein which is about a 6-kDa. Human EGF gene encodes preproprotein that is proteolytically processed to generate a peptide that functions to stimulate the division of epidermal and other cells. Human recombinant VEGFA is commercially available in the form of a 54 amino acid protein having the following sequence:

(SEQ ID NO: 3)
MNSDSECPLSHDGYCLHDGVCMYIEALDKYACNCVVGYIGERCQYRDLKWW
ELR.

The terms, “vascular endothelial growth factor A” or “VEGFA” refers to a heparin-binding protein, which exists as a disulfide-linked homodimer that induces proliferation and migration of vascular endothelial cells. Human recombinant VEGFA is commercially available in the form of a 165 amino acid protein having the following sequence:

(SEQ ID NO: 4)
APMAEGGGQNHHEVVKFMDVYQRSYCHPIETLVDIFQEYPDEIEYIFKPSC
VPLMRCGGCCNDEGLECVPTEESNITMQIMRIKPHQGQHIGEMSFLQHNKC
ECRPKKDRARQENPCGPCSERRKHLFVQDPQTCKCSCKNTDSRCKARQLEL
NERTCRCDKPRR.

The terms, “basic fibroblast growth factor” or “bFGF” refers to protein that has the (3-trefoil structure which binds to FGF receptor (FGFR) family members. Human recombinant bFGF is commercially available in the form of a 154 amino acid protein having the following sequence:

(SEQ ID NO: 5)
AAGSITTLPALPEDGGSGAFPPGHFKDPKRLYCKNGGFFLRIHPDGRVDGV
REKSDPHIKLQLQAEERGVVSIKGVCANRYLAMKEDGRLLASKCVTDECFF
FERLESNNYNTYRSRKYTSWYVALKRTGQYKLGSKTGPGQKAILFLPMSAK
S.

Variants of proteins disclosed herein can be easily produced by a skilled artisan. One can predict functioning variants with structural similarity using computer modeling. Tests confirming inherent activity can be done using procedures outlined in the literature or in this specification. A skilled artisan would understand that one could produce a large number of operable variants that would be expected to have the desirable properties. Genes are known and members share significant homologies from one species to another. The sequences are not identical as illustrated by the differences between the human and mouse sequences SEQ ID NO: 1 and 2 shown in FIG. 6. The sequences are not identical as illustrated by the differences between the human and mouse sequences disclosed in the specification. Only 232 out of 344 amino acids (67%) are identical. Some are conserved substitutions (plus sign). Some are not conserved substitutions. In order to create functioning variants, skilled artisans would not blindly try random combinations, but instead utilize computer programs to make stable substitutions. Skilled artisans would know that certain conserved substations would be desirable. In addition, a skilled artisan would not typically alter evolutionary conserved positions. See Saldano et al. Evolutionary Conserved Positions Define Protein Conformational Diversity, PLoS Comput Biol. 2016, 12(3):e1004775.

Guidance in determining which and how many amino acid residues may be substituted, inserted or deleted without abolishing biological activity may be found using computer programs in combination with publicly available databases well known in the art, for example, RaptorX, ESyPred3D, HHpred, Homology Modeling Professional for HyperChem, DNAStar, SPARKS-X, EVfold, Phyre, and Phyre2 software. See Kelley et al. which report the Phyre2 web portal for protein modelling, prediction and analysis. Nat Protoc. 2015, 10(6):845-58. See also Marks et al., Protein structure from sequence variation, Nat Biotechnol, 2012, 30(11):1072-80; Mackenzie et al. Curr Opin Struct Biol, 2017, 44:161-167; Mackenzie et al. Proc Natl Acad Sci USA. 113(47):E7438-E7447 (2016) and Wei et al. Int. J. Mol. Sci. 2016, 17(12), 2118.

In certain embodiments, this disclosure contemplates using variants of polypeptide sequences disclosed herein having greater than 50%, 60%, 70%, 80%, 90%, 95%, or more identity. “Sequence identity” refers to a measure of relatedness between two or more nucleic acids or proteins, and it is typically given as a percentage with reference to the total comparison length. Identity calculations take into account those amino acid residues that are identical and in the same relative positions in their respective larger sequences. Calculations of identity may be performed by algorithms contained within computer programs such as “GAP” (Genetics Computer Group, Madison, Wis.) and “ALIGN” (DNAStar, Madison, Wis.) using default parameters. In certain embodiments, sequence “identity” refers to the number of exactly matching residues (expressed as a percentage) in a sequence alignment between two sequences of the alignment. In certain embodiments, percentage identity of an alignment may be calculated using the number of identical positions divided by the greater of the shortest sequence or the number of equivalent positions excluding overhangs wherein internal gaps are counted as an equivalent position. For example, the polypeptides GGGGGG (SEQ ID NO: 6) and GGGGT (SEQ ID NO: 7) have a sequence identity of 4 out of 5 or 80%. For example, the polypeptides GGGPPP (SEQ ID NO: 8) and GGGAPPP (SEQ ID NO: 9) have a sequence identity of 6 out of 7 or 85%.

In certain embodiments, for any contemplated percentage sequence identity, it is also contemplated that the sequence may have the same percentage or more of sequence similarity. Percent “similarity” is used to quantify the extent of similarity, e.g., hydrophobicity, hydrogen bonding potential, electrostatic charge, of amino acids between two sequences of the alignment. This method is similar to determining the identity except that certain amino acids do not have to be identical to have a match. In certain embodiments, sequence similarity may be calculated with well-known computer programs using default parameters. Typically, amino acids are classified as matches if they are among a group with similar properties, e.g., according to the following amino acid groups: Aromatic—F Y W; hydrophobic—A V I L; Charged positive: R K H; Charged negative—D E; Polar—S T N Q.

“Subject” means any animal, but is preferably a mammal, such as, for example, a human, monkey, mouse, or rabbit.

As used herein, the terms “treat” and “treating” are not limited to the case where the subject (e.g. patient) is cured and the disease is eradicated. Rather, embodiments of the present disclosure also contemplate treatment that merely reduces symptoms, and/or delays disease progression.

The term “nucleic acid” refers to a polymer of nucleotides or a polynucleotide. The term is used to designate a single molecule, or a collection of molecules. Nucleic acids may be single stranded or double stranded, and may include coding regions and regions of various control elements, as described below.

The term “a polynucleotide having a nucleotide sequence encoding a gene” or “a nucleic acid sequence encoding” a specified polypeptide refers to a nucleic acid sequence comprising the coding region of a gene or in other words the nucleic acid sequence which encodes a gene product. The coding region may be present in either a cDNA, genomic DNA or RNA form. When present in a DNA form, the oligonucleotide, polynucleotide, or nucleic acid may be single-stranded (i.e., the sense strand) or double-stranded. Suitable control elements such as enhancers/promoters, splice junctions, polyadenylation signals, etc. may be placed in close proximity to the coding region of the gene if needed to permit proper initiation of transcription and/or correct processing of the primary RNA transcript. Alternatively, the coding region utilized in the expression vectors of the present invention may contain endogenous enhancers/promoters, splice junctions, intervening sequences, polyadenylation signals, etc. or a combination of both endogenous and exogenous control elements.

The terms “in operable combination”, “in operable order” and “operably linked” refer to the linkage of nucleic acid sequences in such a manner that a nucleic acid molecule capable of directing the transcription of a given gene and/or the synthesis of a desired protein molecule is produced. The term also refers to the linkage of amino acid sequences in such a manner so that a functional protein is produced.

The term “regulatory element” refers to a genetic element which controls some aspect of the expression of nucleic acid sequences. For example, a promoter is a regulatory element which facilitates the initiation of transcription of an operably linked coding region. Other regulatory elements are splicing signals, polyadenylation signals, termination signals, etc.

Promoters may be constitutive or regulatable. The term “constitutive” when made in reference to a promoter means that the promoter is capable of directing transcription of an operably linked nucleic acid sequence in the absence of a stimulus (e.g., heat shock, chemicals, light, etc.). Typically, constitutive promoters are capable of directing expression of a transgene in substantially any cell and any tissue. In contrast, a “regulatable” or “inducible” promoter is one which is capable of directing a level of transcription of an operably linked nuclei acid sequence in the presence of a stimulus (e.g., heat shock, chemicals, light, etc.) which is different from the level of transcription of the operably linked nucleic acid sequence in the absence of the stimulus.

The enhancer and/or promoter may be “endogenous” or “exogenous” or “heterologous.” An “endogenous” enhancer or promoter is one that is naturally linked with a given gene in the genome. An “exogenous” or “heterologous” enhancer or promoter is one that is placed in juxtaposition to a gene by means of genetic manipulation (i.e., molecular biological techniques) such that transcription of the gene is directed by the linked enhancer or promoter. For example, an endogenous promoter in operable combination with a first gene can be isolated, removed, and placed in operable combination with a second gene, thereby making it a “heterologous promoter” in operable combination with the second gene. A variety of such combinations are contemplated (e.g., the first and second genes can be from the same species, or from different species.

Direct Reprogramming of Human Urine Cells into Reprogrammed Vascular Tissue (rVT)

Ischemic cardiovascular diseases, which includes coronary artery disease (e.g., myocardial infarction) and peripheral artery disease (e.g., critical limb ischemia), are a frequent cause of morbidity and mortality. The main cause of these clinical outcomes is the loss of blood vessels. Endothelial cells (ECs), as a key element of vasculature, are indispensable for repairing injured or ischemic tissues. Several approaches have been developed to generate ECs for use in cell therapy. One approach for generating ECs is through direct lineage reprogramming with a single transcription factor (TF) ETV2 (gene or gene products including modified mRNA, proteins, protein containing exosomes), which is specific and crucial for EC development. This strategy has been highlighted due to its potential advantages including a simpler process and avoidance of potential tumorigenicity raised by the usage of pluripotent stem cells (PSCs) and induced pluripotent stem cells (iPSCs). The reprogramming approach outlined herein can be applied to autologous cell therapy.

An enhanced reprogramming method was developed which utilizes EC maturation within short period of time that results in the formation of vascular mimetic tissue including smooth muscle cells. Source cells are collected from patient in a non-invasive manner which can be utilized in clinical applications. By using human urine cells, one can avoid pain associated with sampling source cells by biopsy for dermal fibroblast from the skin. The urine cells are replicated and transformed into an endothelial like and smooth muscle like tissues useful for treating diseases requiring revascularization including, but not limited to, coronary artery diseases, myocardial infarction, heart failure, peripheral artery diseases, critical limb ischemia, stroke, diabetic complications, and would healing. The transformation of human urine cells into vascular mimetic tissue is achieved using protocol which includes specialized culture conditions and delivery of ETV2 gene into human urine cells.

Endogenous synthesis of natural bioscaffold or biomatrix from an autologous source enhances survival of transplanted cells at the target tissue. The natural ECM deposits growth factors released from the therapeutic cells and provides a suitable microenvironment for the cells to initiate neovascularization.

Spontaneously formed natural biomatrix from the therapeutic cellular components present an effective way of generating tissue while bypassing the complicated processes needed for individual cell generation and artificial tissue construction. The natural biomatrix is more biocompatible since it is generated from the autologous source, and the generation mechanism has least risk of pathogen transfer during manufacturing processes. The cell mediated matrix synthesis was not substantially investigated, but the studies indicate that the VSMCs with synthetic phenotype have a critical role in natural matrix formation. Introduction of TGFB and PDGFB induced collagen synthesis. VSMCs under lactate culture medium also had significantly higher collagen synthesis, indicating that the glucose metabolism may affect synthetic phenotype of the cells. Administration of ascorbic acid stimulated collagen biosynthesis from VSMCs and skin fibroblasts. Ascorbic acid, which is a cofactor for hydroxylproline and hydroxylysine, plays a key role in alpha peptide cross-linking during collagen biosynthesis.

Derivation of Urine Cells from Human Urine:

To obtain urine cells from samples, urine is collected and centrifuged. Concentrated urine cells are collected and suspended in growth media which includes EGF, hydrocortisone, epinephrine and fetal bovine serum (FBS). The cell suspension is seeded on gelatin pre-coated cell culture plate and incubated at 37° C., CO₂ (5%) for 14 days. Urine cells form colonies which are maintained to further sub-culture as source cells for direct reprogramming. FBS can be replaced with human serum from a patient for clinical purposes providing for xeno free condition.

Reprogramming of Human Urine Cells into Vascular-Mimetic Issue/Reprogrammed Vascular Tissue (rVT)

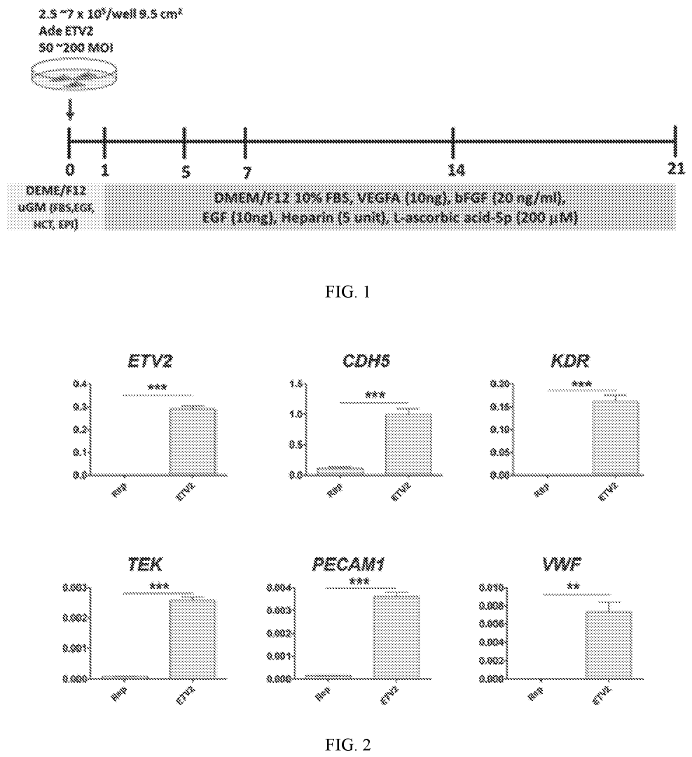

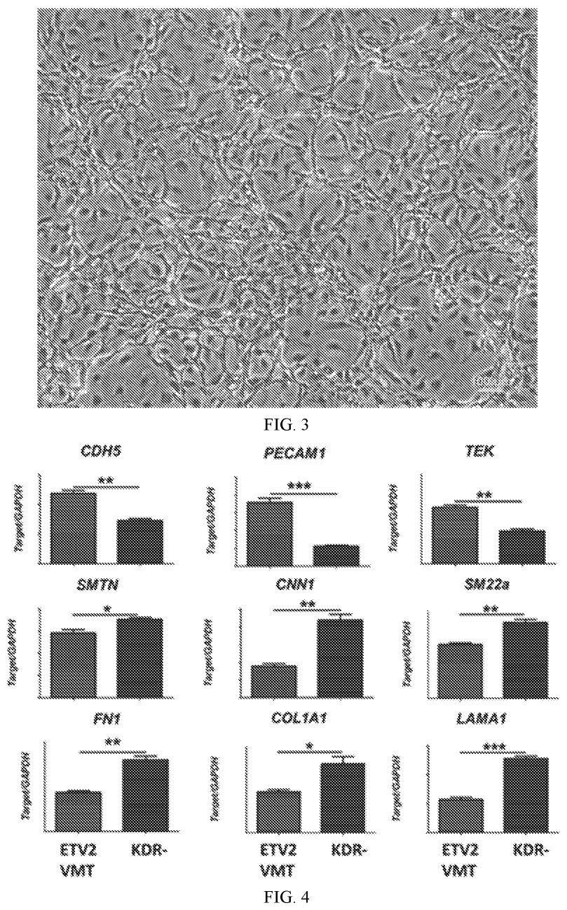

To generate a vascular mimetic tissue, human urine cells are reprogrammed into endothelial cells via overexpression of ETV2 in under specific culture conditions. The reprogramming of human urine cells is initiated through infection of the cells with of an adenovirus that expressed ETV2 in the urine cell growth media for 24 hours. Thereafter the endothelial reprogramming media is changed to include VEGFA, EGF, bFGF, heparin and vitamin C (See FIG. 1). After transduction of ETV2, morphology of urine cells appears as corbel stone shaped endothelial cells. The reprogramming process was monitored by transition of gene profiles and protein expression compared with non-reprogrammed control urine cells. To determine whether urine cells reprogram into endothelial cells, levels of mRNA were measured for endothelial specific genes, such as KDR, CDH5, PECAM1, and VWF, via quantitative real time PCR (FIG. 2). EC genes were significantly induced in ETV2 treated urine cells. In addition to early endothelial genes, late mature endothelial genes such as PECAM1 and VWF were expressed (FIG. 2). Therefore, the induction of EC markers was rapidly increased and reached higher numbers of mature endothelial surface marker such as PECAM1 which does not occur in other types of source cells such as dermal fibroblast. Human dermal fibroblasts do not express such a higher level of PECAM1 within a short reprogramming period of less than 14 days via ETV2 overexpression. Urine derived cells were also trans-differentiated into several lineages such as smooth muscle like cells.

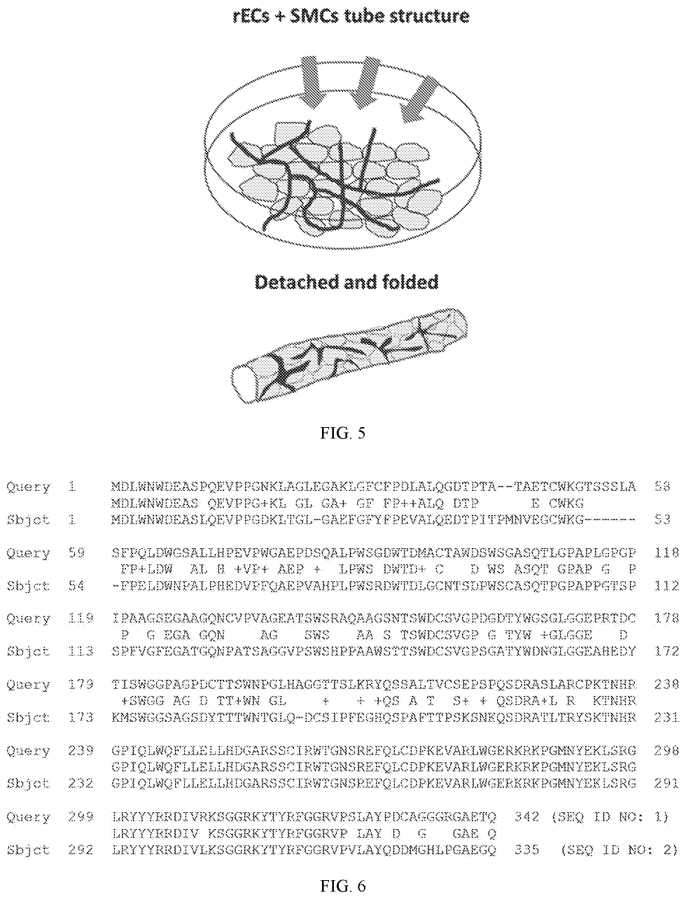

During the endothelial reprogramming, urine cells formed specialized morphology of tube-like structures on the top of cell sheet. To test functional characteristics, uptake of EC specific acetic LDL and binding of UEA1 lectin by was confirmed fluorescence cytochemistry. This tissue co-stained with EC markers and smooth muscle markers such as CDH5, PECAM1, ACTA2 and CNN1. Reprogrammed tissues were enzymatically digested and separated into populations for EC and non-EC using an EC specific marker, KDR. Separated populations via EC (ETV-VMT) versus non-EC markers demonstrated that the reprogrammed tissue was enriched with endothelial and smooth muscle like cells respectively (FIG. 4). The reprogrammed tissues could be mechanically harvested and folded for vascular mimetic structure (FIG. 5). This tissue contained 30-50% of EC markers expressing cells such as KDR, CDH5 and PECAM1. Compare to reprogramming of human dermal fibroblast with ETV2 induction, mature EC marker, PECAM1 is significantly higher and efficiently induced with a shorter reprogramming period. Non-EC marker expressing cells (KDR negative) show high levels of smooth muscle markers such as SM22a, SMTN, ACTA2 and CNN1 (FIG. 4). The tissue containing reprogrammed endothelial and smooth muscle like cells was implanted into mouse hindlimb ischemia models. This tissue was retained in vivo longer than 3 months. Therefore, this tissue is more favorable for direct transplantation or injection into ischemic tissue which needs high retention and cell survival during therapy.

Generating endothelial cells using a variety of other protocols, such as differentiation of PSCs and direct reprogramming of somatic cells, has not been reported to achieved sufficient numbers of functional endothelial cells within a period of time desirable for clinical applications. A major hurdle in using reprogrammed ECs for clinical applications has been poor retention efficiencies of injected ECs into the ischemic target areas of patients. The methods disclosed herein have several advantages compare with other EC generation techniques. Urine provides limitless supply of source cells that can be obtained by non-invasive manner. EC generation is more timely compare other methods. One obtains reprogrammed endothelial and smooth muscle cells together within a cell sheet structure that has good retention in ischemic areas without foreign biomaterials. This reprogrammed vascular mimetic tissue is applicable for autologous cell therapy in various ischemic disease such as PAD and MI.

1. A method of producing endothelial and smooth muscle like vascular tissue comprising:
i) concentrating urine cells from a subject;
ii) replicating the concentrated urine cells in a first growth media comprising
a) EGF,
b) hydrocortisone,
c) epinephrine and
d) human serum or animal serum;
providing purified concentrated urine derive cells;
iii) exposing the purified concentrated urine derive cells to ETV2;
iv) culturing the purified concentrated urine derive cells in the first growth media providing endothelial like urine derived cells;
v) culturing the endothelial like urine derive cells in a second growth media comprising:
a) EGF,
b) VEGFA,
c) bFGF,
d) heparin,
e) L-ascorbic acid, and
d) human serum or animal serum;
providing endothelial and smooth muscle like vascular tissue.

2. The method of claim 1, wherein the human serum is from the subject.

3. The method of claim 1, wherein exposing the purified concentrated urine derive cells to ETV2 is by mixing the purified concentrated urine derived cells with a recombinant virus that infects the purified concentrated urine derive cells, comprises a gene encoding ETV2, and expresses ETV2 after infection.

4. The method of claim 3, wherein the recombinant virus is an adenovirus or lentivirus.

5. The method of claim 1, wherein the growth media comprises glucose, amino acids, and vitamins, glutamine, and sodium pyruvate.

6. The method of claim 1, further comprising the step of folding the endothelial and smooth muscle like vascular tissue into a three-dimensional structure.

7. The method of claim 1, further comprising implanting the endothelial and smooth muscle like vascular tissue into the subject.

8. The method of claim 7, wherein implanting the endothelial and smooth muscle like vascular tissue is by contacting the endothelial and smooth muscle like vascular tissue with a vein, artery, capillary, heart muscle, skin, lung, kidney or intestine.


SEQUENCE LISTING (9 sequences)

SEQ ID NO: 1 - protein, 342 aa, Homo sapiens
MDLWNWDEASPQEVPPGNKLAGLEGAKLGFCFPDLALQGDTPTATAETCWKGTSSSLASFPQLDWGSALLHPEVPWGAEPDSQALPWSGDWTDMACTAWDSWSGASQTLGPAPLGPGPIPAAGSEGAAGQNCVPVAGEATSWSRAQAAGSNTSWDCSVGPDGDTYWGSGLGGEPRTDCTISWGGPAGPDCTTSWNPGLHAGGTTSLKRYQSSALTVCSEPSPQSDRASLARCPKTNHRGPIQLWQFLLELLHDGARSSCIRWTGNSREFQLCDPKEVARLWGERKRKPGMNYEKLSRGLRYYYRRDIVRKSGGRKYTYRFGGRVPSLAYPDCAGGGRGAETQ

SEQ ID NO: 2 - protein, 335 aa, Mus musculus
MDLWNWDEASLQEVPPGDKLTGLGAEFGFYFPEVALQEDTPITPMNVEGCWKGFPELDWNPALPHEDVPFQAEPVAHPLPWSRDWTDLGCNTSDPWSCASQTPGPAPPGTSPSPFVGFEGATGQNPATSAGGVPSWSHPPAAWSTTSWDCSVGPSGATYWDNGLGGEAHEDYKMSWGGSAGSDYTTTWNTGLQDCSIPFEGHQSPAFTTPSKSNKQSDRATLTRYSKTNHRGPIQLWQFLLELLHDGARSSCIRWTGNSREFQLCDPKEVARLWGERKRKPGMNYEKLSRGLRYYYRRDIVLKSGGRKYTYRFGGRVPVLAYQDDMGHLPGAEGQ

SEQ ID NO: 3 - protein, 54 aa, Artificial
  Synthetic construct
MNSDSECPLSHDGYCLHDGVCMYIEALDKYACNCVVGYIGERCQYRDLKWWELR

SEQ ID NO: 4 - protein, 165 aa, Artificial
  Synthetic construct
APMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQENPCGPCSERRKHLFVQDPQTCKCSCKNTDSRCKARQLELNERTCRCDKPRR

SEQ ID NO: 5 - protein, 154 aa, Artificial
  Synthetic construct
AAGSITTLPALPEDGGSGAFPPGHFKDPKRLYCKNGGFFLRIHPDGRVDGVREKSDPHIKLQLQAEERGVVSIKGVCANRYLAMKEDGRLLASKCVTDECFFFERLESNNYNTYRSRKYTSWYVALKRTGQYKLGSKTGPGQKAILFLPMSAKS

SEQ ID NO: 6 - protein, 6 aa, Artificial
  Synthetic construct
GGGGGG

SEQ ID NO: 7 - protein, 5 aa, Artificial
  Synthetic construct
GGGGT

SEQ ID NO: 8 - protein, 6 aa, Artificial
  Synthetic construct
GGGPPP

SEQ ID NO: 9 - protein, 7 aa, Artificial
  Synthetic construct
GGGAPPP